(19) United States
(12) Patent Application Publication
(10) Pub. No.: US 2004/0251188 A1
(43) Pub. Date: Dec. 16, 2004

(54) Immoblized alkylated amine functional macromolecules, alkylated ammonium salt functional macromolecules, and alkylated quaternary ammonium salt functional macromolecules, process for their preparation and methods for their use

(76) Inventors: Charles E. Skinner, Midland, MI (US); Yung K. Kim, Midland, MI (US); William H. Campbell, Midland, MI (US)

Correspondence Address: MCKELLAR STEVENS & HILL PLLC, POSEYVILLE PROFESSIONAL COMPLEX, 784 SOUTH POSEYVILLE ROAD, MIDLAND, MI, 48640, US

(21) Appl. No.: 10/458,114

(22) Filed: Jun. 10, 2003

(51) Int. Cl. B01D 15/08

(52) U.S. Cl. 210/198.2; 210/502.1

(57) ABSTRACT

Alkylated amine functional macromolecules, ammonium salt functional macromolecules, and quaternized ammonium salt functional macromolecules that are bonded to solid substrates, processes for their preparation, and methods for their use in liquid chromatography, process stream purification, waste stream clean up, resource recovery and personal care products, among other uses.

This invention deals with alkylated amine functional macromolecules, alkylated ammonium salt functional macromolecules and alkylated quaternary ammonium salt functional macromolecules, the derivatized macromolecules being tethered to a silica or other substrate; and processes for their preparation, and methods for their use in liquid chromatography, process stream purification, waste stream clean up, resource recovery and personal care products, among others.

### BACKGROUND OF THE INVENTION

Since the decade of the nineteen eighties there has been a large volume of information reported on macromolecules, most specifically, the macromolecules that are dendritic in nature, and those that are hyperbranched.

Dendrimers are described as globular, nano-scale macromolecules consisting of two or more tree-like dendrons, emanating from a single central atom or atomic group called the core. They are comprised of branch cells that are the main building blocks of dendritic structures; that is, three-dimensional analogues of the repeating units in classical linear polymers; that must contain at least one branch juncture, and that are organized in geometrically precise architectural arrangements, that give rise to a series of regular radially concentric layers, called generations (G) around the core. Dendrimers must contain at least three different types of branch cells including a core, interior cells, and surface or exterior cells.

Dendrons are the smallest constitutive elements of a dendrimer that have the same architectural arrangement as the dendrimer itself, but which emanate from a single trunk or branch, which may end with a potentially reactive, or a potentially inert functional group called by those skilled in this particular art, the focal group.

On the other hand, hyperbranched polymers are random, highly branched macromolecules usually obtained from a “one-shot” polymerization reaction of an AB, type of monomer, that is nAB_{w}→----(AB_{w})ₙ------, where A and B represent mutually reactive functional groups of the monomer. They are usually different from dendrons, in that, hyperbranched macromolecules are considerably more architecturally variable in their structure; have a lower degree of branching; and as materials, usually have a high degree of polydispersity, in that, not all hyperbranched macromolecules of the same hyperbranched polymer are of the same molecular weight or chain length.

A pictorial representation showing in detail the proposed architecture of these types of macromolecular structures can be found in Polymer Preprints, Division of Polymer Chemistry, American Chemical Society, Volume 39, Number 1, Pages 473 to 474, (March, 1998).

Most recently, there has been provided a new set of amine functional macromolecules, that is, multi-layered macromolecules that are covalently bonded to solid particulate substrates to immobilize them. The multi-layered macromolecules consist of a base macromolecule that is selected from the group consisting of dendrimers and hyperbranched polymers, analogous to those described just above, and then, additional layers of bonded macromolecules are chemically bonded to the base macromolecule also selected from the dendrimers or hyperbranched polymers, or combinations of them.

The bonding of the macromolecules to the solid particulate substrates is undertaken as described herein using the same or similar silane bonding agents as described herein, and the bonding of one layer of macromolecule to another layer of macromolecule for purposes of this invention can also be undertaken by using such silane bonding agents.

With the exception of the multi-layered macromolecules, much of the detail of these polymers, their chemical reactions schemes, their combinations, and some of their intended and proposed uses can be found in U.S. Pat. No. 5,739,218 that issued to Dvornic, et al. on Apr. 14, 1998; U.S. Pat. No. 5,902,863 that issued to Dvornic, et al. on May 11, 1999; U.S. Pat. No. 5,938,934 that issued to Balogh on Aug. 17, 1999 and U.S. Pat. No. 6,077,500 that issued to Dvornic on Jun. 20, 2000, all of which are incorporated herein by reference for what they teach about the polymers and the methods by which they are made.

Dvornic, et al., in U.S. Pat. Nos. 5,902,863, U.S. Pat. No. 5,739,218, and U.S. Pat. No. 6,077,500 and Balogh, et al., teach the preparation of organosilicon macromolecules that are based on dendrimer networks that are prepared from radially layered polyamido-amine-organosilicon (PAMAMOS) or polypropyleneimine-organosilicon (PPIOS) dendrimer precursors. The silicon-containing networks have covalently bonded hydrophilic and hydrophobic nanoscopic domains whose size, shape, and relative distribution can be precisely controlled by the reagents and conditions disclosed therein. The PAMAMOS or PPIOS dendrimers can be cross linked into dendrimer-based networks by any number of different types of reactions. For example, Dvornic, et al., in U.S. Pat. No. 5,739,218 teaches hydrophilic dendrimers whose surface has been partially or completely derivatized with inert or functional organosilicon moieties.

Further, Dvornic, et al., in U.S. Pat. No. 6,077,500 teach reacting organosilicon compounds with macromolecules including a higher generation of radially layered copolymeric dendrimers having hydrophilic polyamidoamine or a hydrophilic polypropyleneimine interior and a hydrophobic organosilicon exterior. Balogh et al., teach dendritic polymer based networks that consist of hydrophilic and oleophobic domains.

The general applications for the materials of the above-mentioned patents are for coatings, sensors, sealants, insulators, conductors, absorbents, delivering active species to specific areas such as in catalyst use, drug therapy and gene therapy, personal care uses, and agricultural adjuvant products.

A more recent, somewhat related disclosure utilizing a polyamine as the base polymer can be found in Rosenberg, U.S. Pat. No. 5,695,882 that issued on Dec. 9, 1997 wherein there is disclosed a system for extracting soluble heavy metals from liquid solutions. The process makes use of an activated surface of an extraction material that is a reaction product of an unbranched polyamine with a covalently anchored trifunctional hydrocarbyl silyl that yields non-crosslinked amino groups to which functional chelator groups can be covalently attached. The activated surface of the extraction material is formed by first hydrating the extraction material surface and then silanizing the hydrated surface with a short chain trifunctional silane having a hydrocarbon substituent containing 1 to 6 carbon atoms and a terminal leaving group, and then reacting a polyamine with the hydrocarbysilyl from the silanization of the hydrated surface so as to form an aminohydrocarbyl polymer covalently bound to the extraction material surface.

It should be noted that this material is not polymeric and therefore, is non-crosslinked as is expressly stated therein by the patentees. U.S. Pat. No. 5,087,359 that issued to Kakodkar, et al. on Feb. 11, 1992 discloses the quaternization of the non-crosslinked polyethyleneimine silica based solid supported materials and also discloses them as being useful for strong anion exchangers for column chromatography separation and purification of proteins among other uses.

Another U.S. patent, namely, U.S. Pat. No. 5,997,748, that issued on Dec. 7, 1999 to Rosenberg and Pang, teaches essentially the same technology as is set forth in the earlier Rosenberg patent as this latter patent is a divisional application from the earlier patent.

What these references do not teach are the inventive polymeric compositions disclosed herein, processes for the preparation of the inventive compositions, and the applications for the use of the inventive compositions of this invention as described and claimed herein.

### BRIEF DESCRIPTION OF THE DRAWINGS

FIG. 1 depicts the reaction of a silica substrate with a bonding silane of this invention to form the silane bonded on the silica substrate.

FIG. 2 depicts the reaction of the unreacted portion of the bonding silane with a amine functional macromolecule to bond the macromolecule to the solid substrate.

FIG. 3 depicts the reaction of the bonded macromolecule via the Eschweiler-Clarke reaction to form an alkylated amine of this invention.

FIG. 4 depicts the reaction of the bonded macromolecule using a methyl halide to fully quaternize the macromolecule.

FIG. 5 depicts the ammonium salt formed by the addition of a strong acid to the amine.

### THE INVENTION

A first embodiment of this invention is an alkylated amine functional macromolecule comprising a base macromolecule covalently bonded to a solid substrate wherein the base macromolecule is selected from the group consisting of amine functional (a) dendrimers, (b) hyperbranched polymers, (c) multilayered dendrimers, (d) multilayered, hyperbranched polymers, (e) multilayered polymers of mixed dendrimer, and hyperbranched multilayers. The amine functionality of the macromolecule is further modified to provide alkylated amine functionality.

A second embodiment of this invention is an ammonium salt functional macromolecule comprising a base macromolecule covalently bonded to a solid substrate wherein the base macromolecule is selected from the group consisting of amine functional (a) dendrimers, (b) hyperbranched polymers, (c) multilayered dendrimers, (d) multilayered hyperbranched polymers, (e) multilayered polymers of mixed dendrimer, and hyperbranched multilayers. The amine functionality of the macromolecule is further modified either with an acid to form an ammonium salt functional macromolecule directly from the amine functional macromolecule.

A third embodiment of this invention is an alkylated ammonium salt functional macromolecule comprising a base macromolecule covalently bonded to a solid substrate wherein the base macromolecule is selected from the group consisting of amine functional (a) dendrimers, (b) hyperbranched polymers, (c) multilayered dendrimers, (d) multilayered hyperbranched polymers, (e) multilayered polymers of mixed dendrimer, and hyperbranched multilayers. The amine functionality of an alkylated macromolecule is further modified by treating the compositions of matter from the first embodiment with an acid to form an alkylated ammonium salt functional macromolecule.

Yet, a fourth embodiment of this invention is a quaternary ammonium salt functional macromolecule comprising a base macromolecule covalently bonded to a solid substrate wherein the base macromolecule is selected from the group consisting of amine functional (a) dendrimers, (b) hyperbranched polymers, (c) multilayered dendrimers, (d) multilayered hyperbranched polymers, (e) multilayered polymers of mixed dendrimer, and hyperbranched multilayers. The amine functionality of the macromolecule is further modified to provide alkylated amine functionality that is further alkylated to provide a quaternary ammonium salt.

Still a fifth embodiment of this invention is a process for preparing an alkylated amine functional macromolecule, the process comprising (I) providing a solid substrate capable of reacting with a silane bonding agent; (II) contacting the solid substrate with a silane bonding agent and allowing the solid substrate to react with the silane bonding agent; (III) contacting the product formed in step (II) with a base macromolecule selected from the group consisting of amine functional (a) dendrimers, (b) hyperbranched polymers, (c) multilayered dendrimers, (d) multilayered hyperbranched polymers, (e) multilayered polymers of mixed dendrimer, and hyperbranched multilayers and allowing the base macromolecule and the product from step (II) to react with each other; (IV) contacting the product from step (III) with formic acid and formaldehyde in a solvent at a temperature in excess of ambient temperature to form an alkylated amine functional macromolecule.

Yet another embodiment of this invention is a process for preparing an ammonium salt functional macromolecule, the process comprising (I) providing a solid substrate capable of reacting with a silane bonding agent; (II) contacting the solid substrate with a silane bonding agent and allowing the solid substrate to react with the silane bonding agent; (III) contacting the product formed in step (II) with a base macromolecule selected from the group consisting of amine functional (a) dendrimers, (b) hyperbranched polymers, (c) multilayered dendrimers, (d) multilayered hyperbranched polymers, (e) multilayered polymers of mixed dendrimer and hyperbranched multilayers and allowing the base macromolecule and the product from step (II) to react with each other; (IV) contacting the product from step (III) with formic acid and formaldehyde in a solvent at a temperature in excess of ambient temperature to form an alkylated macromolecule; (V) contacting the product of either step (III) or step (IV) with an acid to from an ammonium salt or an alkylated ammonium salt functional macromolecule, respectively.

Going to still another embodiment of this invention, there is a process for preparing a quaternary ammonium salt functional macromolecule comprising (I) providing a solid substrate capable of reacting with a silane bonding agent; (II) contacting the solid substrate with a silane bonding agent and allowing the solid substrate to react with the silane bonding agent; (III) contacting the product formed in step (II) with a base macromolecule selected from the group consisting of amine functional (a) dendrimers, (b) hyperbranched polymers, (c) multilayered dendrimers, (d) multilayered hyperbranched polymers, (e) multilayered polymers of mixed dendrimer and hyperbranched multilayers and allowing the base macromolecule and the product from step (II) to react with each other; (W) contacting the product from step (III) with formic acid and formaldehyde in a solvent at a temperature in excess of ambient temperature to form an alkylated macromolecule; (V) contacting the alkylated macromolecule with an acid scavenger and an excess of a alkyl halide in the presence of a solvent at ambient temperature or higher for a period of time to form a quaternary ammonium salt functional macromolecule.

Another embodiment of this invention is a process for preparing an alkylated amine functional macromolecule comprising (I) providing a lot of base macromolecule selected from the group consisting of (a) dendrimers, (b) hyperbranched polymers, (c) multilayered dendrimers, (d) multilayered hyperbranched polymers, (e) multilayered polymers of mixed dendrimer and hyperbranched multilayers; (II) contacting the base macromolecule with a silane bonding agent and allowing the silane bonding agent and the base macromolecule to react with each other; (III) contacting the product from step (II) with a solid substrate capable of reacting with the product from step (II); (IV) allowing the product from step (II) to bond to the solid substrate by providing at least one of the conditions selected from the group consisting of (i) initially combining the components in step (II) in the absence of water and thereafter, contacting the combine components with water and, (ii) initially combining the components in step (III) with sufficient water for hydrolysis of any hydrolysable groups in the components and thereafter, adding additional water for crosslinking any silanols formed by the initial water for hydrolysis; (V) contacting the product from step (W) with formic acid and formaldehyde in a quantity selected from the group consisting of (i) substoichiometric, (ii) stoichiometric, and (iii) excess stoichiometric based on the amount of reactive amine in the base macromolecule, in a solvent at a temperature selected from a range of from ambient temperature to higher than ambient temperature, to form an alkylated amine functional macromolecule.

Moving to yet another embodiment of this invention, there is a process for preparing an ammonium salt functional macromolecule, the process comprising (I) providing a lot of base macromolecule selected from the group consisting of (a) dendrimers, (b) hyperbranched polymers, (c) multilayered dendrimers, (d) multilayered hyperbranched polymers, (e) multilayered polymers of mixed dendrimer and hyperbranched multilayers; (II) contacting the base macromolecule with a silane bonding agent and allowing the silane bonding agent and the base macromolecule to react with each other; (III) contacting the product from step (II) with a solid substrate capable of reacting with the product from step (II); (IV) allowing the product from step (III) to bond to the solid substrate by providing at least one of the conditions selected from the group consisting of (i) initially combining the components in step (II) in the absence of water and thereafter, contacting the combine components with water and, (ii) initially combining the components in step (III) with sufficient water for hydrolysis of any hydrolysable groups in the components and thereafter, adding additional water for crosslinking any silanols formed by the initial water for hydrolysis; (V) contacting the product from step (IV) with formic acid and formaldehyde in a quantity selected from the group consisting of (i) substoichiometric, (ii) stoichiometric, and (iii) excess stoichiometric based on the desired level of alkylation of the base macromolecule, in a solvent at a temperature selected from a range of from ambient temperature to higher than ambient temperature, to form an alkylated macromolecule; (V) contacting the product of either step (III) or step (IV) with an acid to form an ammonium salt or an alkylated ammonium salt functional macromolecule, respectively.

There is a final embodiment that is a process for preparing a quaternized ammonium salt functional macromolecule comprising (I) providing a lot of base macromolecule selected from the group consisting of (a) dendrimers, (b) hyperbranched polymers, (c) multilayered dendrimers, (d) multilayered hyperbranched polymers, (e) multilayered polymers of mixed dendrimer and hyperbranched multilayers; (II) contacting the base macromolecule with a silane bonding agent and allowing the silane bonding agent and the base macromolecule to react with each other; (III) contacting the product from step (II) with a solid substrate capable of reacting with the product from step (II); (IV) allowing the product from step (II) to bond to the solid substrate by providing at least one of the conditions selected from the group consisting of (i) initially combining the components in step (II) in the absence of water and thereafter, contacting the combine components with water and, (ii) initially combining the components in step (III) with sufficient water for hydrolysis of any hydrolysable groups in the components and thereafter, adding additional water for crosslinking any silanols formed by the initial water for hydrolysis (V) contacting the product from step (IV) with formic acid and formaldehyde in a quantity selected from the group consisting of (i) substoichiometric, (ii) stoichiometric, and (iii) excess stoichiometric based on the desired level of alkylation of the base macromolecule, in a solvent at a temperature selected from a range of from ambient temperature to higher than ambient temperature, to form an alkylated amine functional macromolecule; (VI) contacting the alkylated macromolecule with an acid scavenger and a stoichiometric excess of a alkyl halide in the presence of a solvent at ambient temperature or higher for a period of time to form a quaternary ammonium salt functional macromolecule.

The base macromolecule in each of these embodiments is selected from the group consisting of amine functional dendrimers, amine functional hyperbranched polymers, and multi-layered amine functional polymers as described herein,

### DETAILED DESCRIPTION OF THE INVENTION

Turning now to the details of the present invention and with specificity, there is disclosed herein a composition of matter that is an alkylated or quaternized amine functional macromolecule that is covalently bonded to a solid substrate.

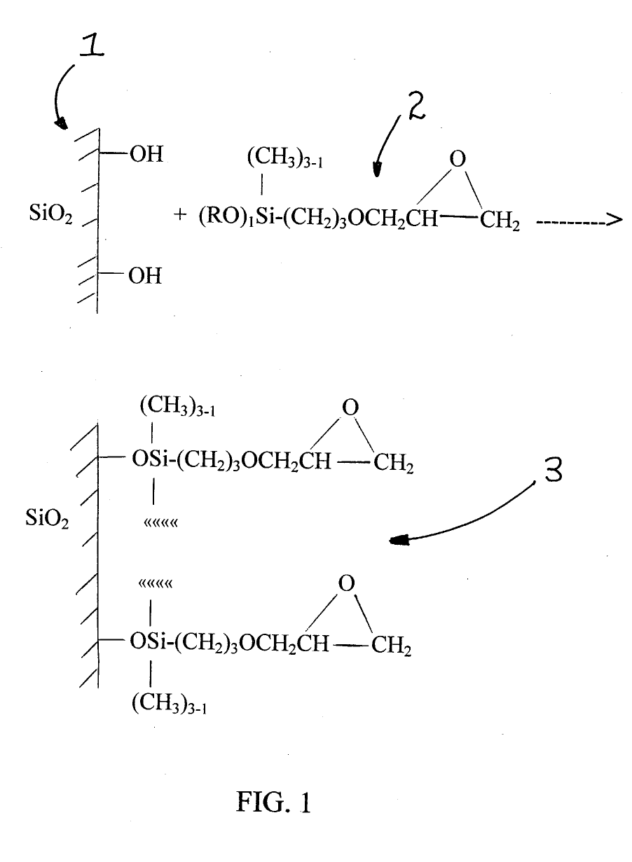

FIG. 1 shows the reaction scheme for treating the solid particulate material, in this case the SiO₂, depicted as 1, an example of the silane bonding agent depicted at 2, and the depiction of the resulting reacted product at 3. It should be noted that the SiO₂ 1 is depicted by way of example, and the silane is 3-glycidoxypropyltrimethoxysilane, by way of example. The <<<<<<<<represent potential siloxane linkages, OH, or OR groups, or other bonding to the solid substrate, depending on the degree of reactivity and process conditions for the reaction scheme.

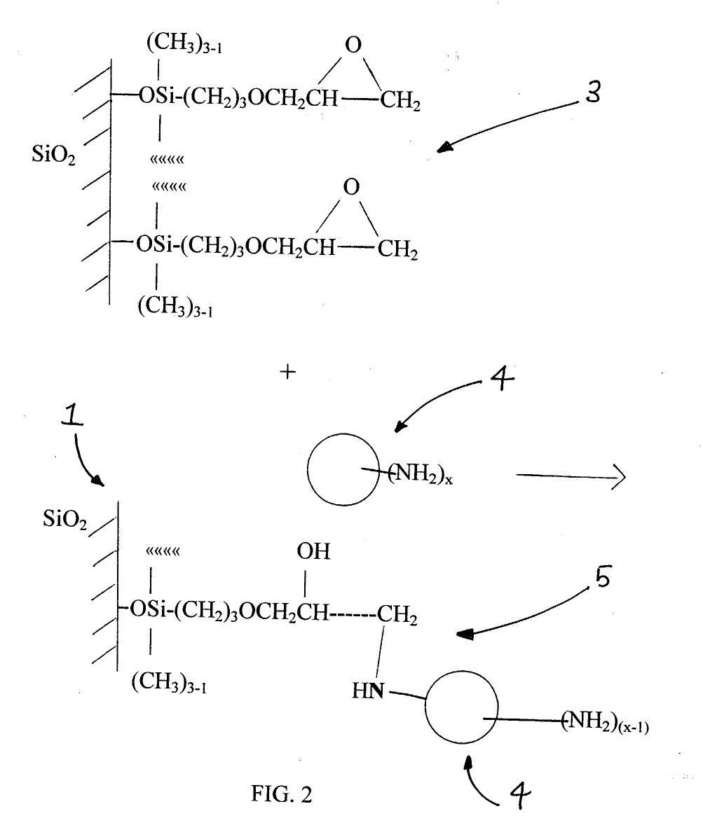

FIG. 2 depicts the reaction of the unreacted portion of the bonding silane with an amine functional macromolecule 4, to bond the macromolecule to the solid substrate. as shown at 5.

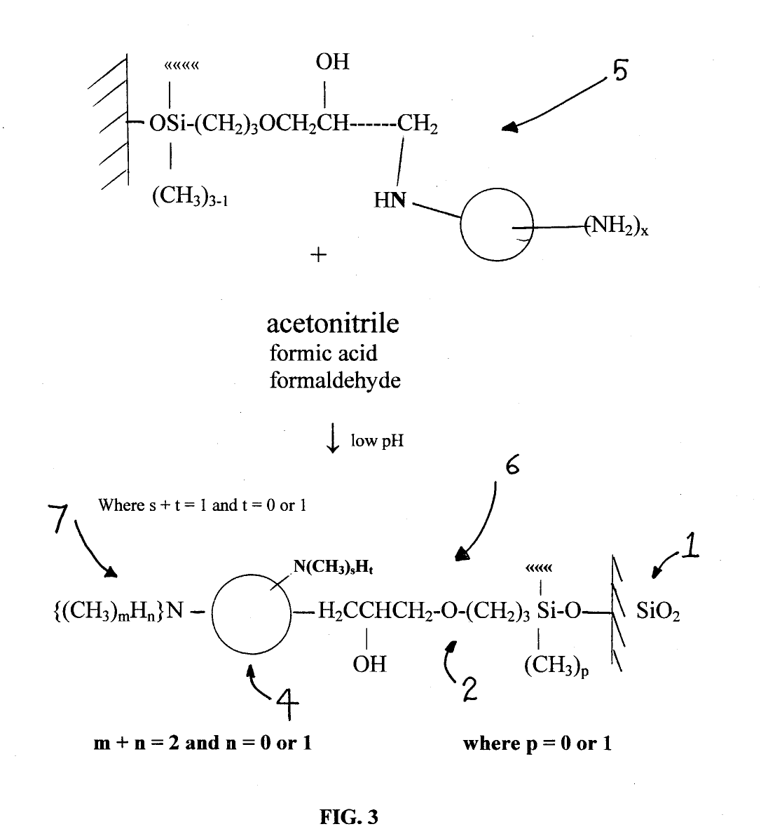

FIG. 3 depicts the reaction of the bonded macromolecule via the Eschweiler-Clarke reaction to form an alkylated amine functional macromolecule of this invention wherein the alkylated end of the molecule is shown at 7.

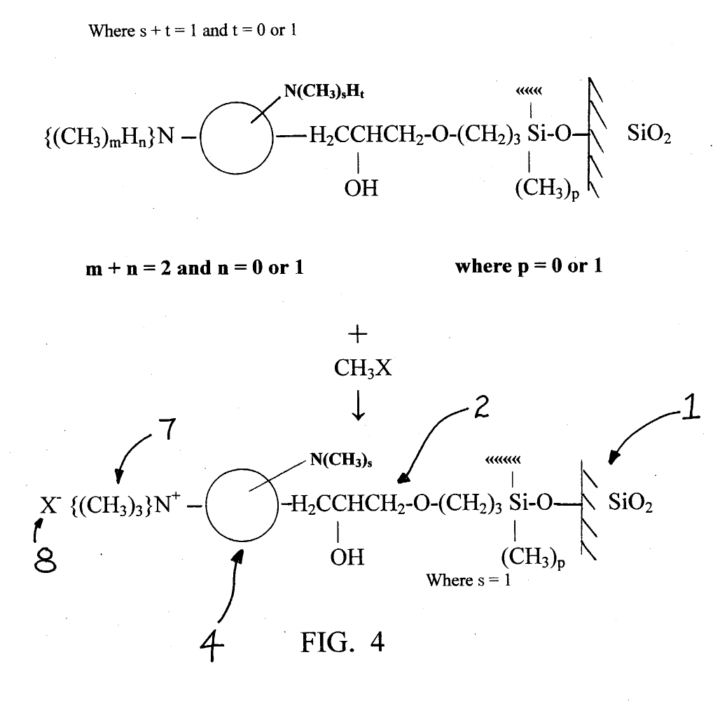

J FIG. 4 depicts the reaction of the bonded macromolecule using a methyl halide to fully quaternize the macromolecule, the quaternization shown at 8.

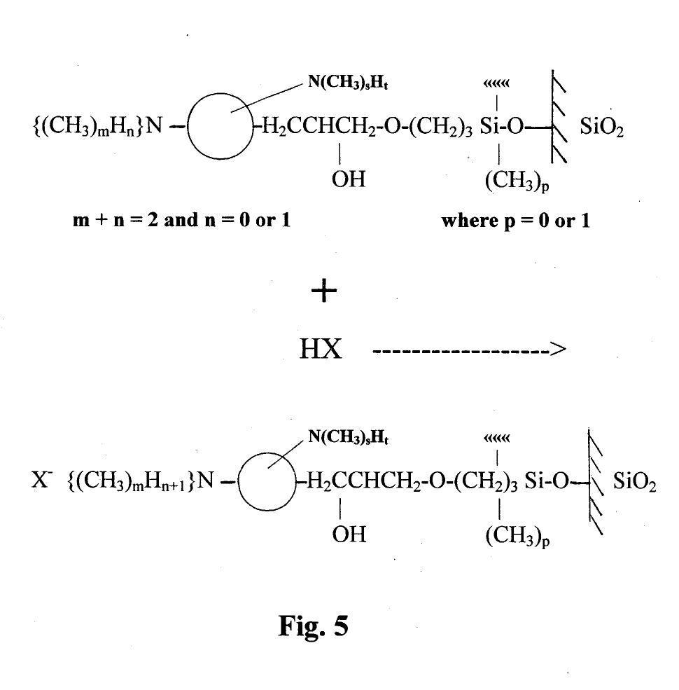

FIG. 5 depicts the ammonium salt formed by the addition of a strong acid to the amine functionality, wherein the salt is shown at 9.

The materials of this invention can be prepared by the use of any alkyl halide having the general formula R³X wherein R³ is an alkyl group consisting of 1 to 18 carbon atoms and X is selected from Cl, Br, F, or I. For purposes of this invention, there can be used a mixture of alkyl halides wherein R³ can be any alkyl group having 1 to 18 carbon atoms such that the macromolecule is substituted with a mixture of alkyl groups, for example, a combination of methyl and hexyl.

Whenever Eschweiler-Clarke is used herein it is intended to be the generic reaction scheme described in “Chemistry of Organic Compounds”, Noller, 2^{nd} edition, W. B. Saunders Company, Philadelphia, Pa., USA, 1957, pp. 231.

It is preferred for this invention to add the silane bonding agent to the silica in the absence of any solvent, however, solvents can be used. Adding the silane bonding agent to the silica in the absence of solvent is referred to herein as dry bonding.

Thus, organic solvents that are useful in the process of this invention are any polar solvent and those solvents that have been found convenient are, for example, methanol, dimethylformamide, dimethylsulfoxide, tetrahydrofuran, acrylonitrile, and the like. Preferred solvents are methanol, dimethylformamide and dimethylsulfoxide. Aprotic polar solvents are best for the amine capture reaction between the epoxy group of the bonding agent and any amine compound.

The amount of time that the reactants in any step of the processes are reacted together is not overly critical, the objective being to alkylate the amine functional polymer to the degree that is desired for the end use application. Thus, time of reaction for each step can range from six hours to about three days and the preferred amount of time for the reaction of the solid substrate with the silane bonding agent is 2.5 to 3 days, while the alkylation reaction is preferred to be carried out over a time range of from about 12 hours to about 18 hours.

The temperature at which the processes of this invention are carried out are not critical, and the temperature range is from about room temperature to about 80° C., depending on the desired objective. Preferred for these processes is a temperature range of from room temperature to about 65° C., and most preferred is a temperature range of from room temperature to about 50° C.

It should be noted that the final step of the process wherein the amine functional macromolecule is quaternized, depends on the amount of alkyl halide that is used in the reaction.

Turning now to the various other components that are useful in this invention, it should be noted that the solid substrates of this invention are any solid substrates that will bond to a silane bonding agent of this invention, for example, films, membranes, and particulate mineral materials, including silica, that provide a stable —SiO— bond when bound to a silane bonding agent of this invention. Included in this group are silicas, including fumed, precipitated, and ground silicas, along with other forms, such as silica gels. The polymeric films and membranes can have the attributes set forth above for particulate substrates, and can in addition, have reactive halogens that can react directly with the amine functional polymers described herein, or amine groups that are reactive to the silane bonding agents as set forth just below.

Also useful are organic resin particles that have reactive halogens that can react directly with the amine functional polymers described herein, or amine groups that are reactive to the silane bonding agents of this invention, such as Dowex® ion exchange beads, and the like. The silane bonding agents of this invention are any functional silane that comprises a hydrolyzable leaving group that allows the reaction of the silane bonding agent with the solid substrate, or, with silanes with functional groups capable of reacting with the organic resin particles containing a reactive halogen group, and which silanes also contain a reactive group capable of reacting with the functional groups of the macromolecules.

The silane bonding agents of this invention preferably have the general formulae

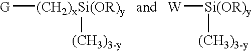

wherein G is selected from the radicals

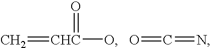

halide, especially Cl, Br, epoxy, and vinyl, and W is selected from ClCH₂CH₂-Phenyl, and wherein x has a value of from 1 to 6 and y has a value of 1, 2, or 3, R is selected from the group consisting of an alkyl group of from 1 to 6 carbon atoms and the phenyl radical.

Also useful silane bonding agents for this invention are the silane bonding agents having the general formula

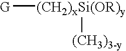

and those silane bonding agents having the formula:

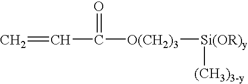

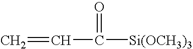

is one of the preferred silane bonding agents, while the silane bonding agent having the general formula

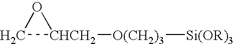

is highly preferred. Most preferred of this general formula is the silane bonding agent having the specific formula:

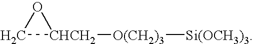

Most preferred of these materials is the aforementioned 3-glycidoxypropyltrimethoxysilane.

The immobilized macromolecules of this invention have many uses. Among them are in methods of analysis wherein the macromolecules are used to separate desired materials from materials associated with the desired material to be separated. For example, the materials of this invention have been found be very useful as fillers in gas chromatography columns for separation techniques using the liquid chromatography process.

The materials of this invention are also useful in methods of process stream purification wherein effluent process streams are treated with the macromolecular compositions to remove certain contaminants and the like. For example, the compositions have been found very useful for removing metals from process streams and they have been found useful for removing acids from certain process streams.

In addition, the materials have been found very useful in treating waste streams of all types to remove certain materials from such waste streams, for example, the removal of metal ions and acids. One very valuable method is the recovery of precious metals from various fluids containing such metals.

For example, any of the following metals or combination of metals can be removed from process streams, waste streams or other effluents: silver, gold, cadmium, chromium, copper, hafnium, iridium, manganese, molybdenum, niobium, osmium, palladium, platinum, rhenium, rhodium, ruthenium, tantalum, technetium, titanium, tungsten, zinc, zirconium and heavy metals such as barium, bismuth, cerium, lead, antimony, tin, thallium, uranium and plutonium.

With regard to acids, the organic acids that have been removed from streams are p-aminobenzoic acid, carboxylic acid, salicylic acid and acetasalicylic acid, among others.

The materials of this invention can act as anion exchange resins and can be used to separate proteins, peptides, and oligionucleotides wherein the separation can be of these materials from other materials, or in the case of proteins, for example, the separation of certain proteins from other proteins, and in the case of peptides, the separation of peptides form other materials and the separation of certain peptides from other peptides, and the like.

It has also been found that the materials of this invention are useful for concentrating anionic materials and such materials are for example, molybdates, arsenates, phosphates, dichromates, tungstates, zirconates, titanates, cerates, vanadates, complex anionic materials, or any combination of these materials.

Finally, it has been discovered that these materials are very useful in personal care products, household care products, health care products, such as, for example, topical health care compositions and in vivo health care compositions.

### EXAMPLES

### Example 1

### Synthesis of a Methylated Polyethyleneimine Bonded to a Silica Surface

Silica, 50 grams, (Daiso SP-300-10P) having a surface area of 108 m²/gram and having a pore diameter of 263 Å and a particle size of 9 μ was suspended in 250 milliliters of toluene and the mixture was azeotroped for one hour to remove water from the silica surface. A silane bonding agent, 3-glycidoxypropyltrimethoxysilane (Silar Laboratories), 5.3 grams, and glacial acetic acid was added to the silica to provide 2.5 molecules/nm² of silane coverage on the silica. The reaction mixture was stirred at 50° C. for three days, and then filtered and washed twice with toluene and twice with methanol to provide silica having the silane bonded to its surface.

This product, 25 grams was suspended in dimethylformamide and then 3.75 grams of polyethyleneimine (BASF, approximately 25,000 molecular weight) was added and the mixture was stirred overnight, and then place in a 40° C. water bath for two hours before washing twice with methanol and once with methanol/water (50/50) and then twice more with methanol. The product was then dried overnight (about 16 hrs.) at 70° C. and sieved to yield 24.1 grams of material. Analysis indicated that the resulting product contained 6% of the polyethyleneimine attached to the silica surface.

The polyethyleneimine on the silica surface contained primary and secondary amine groups and was methylated using the Eschweiler-Clarke procedure, namely, 15 grams of the product from above was suspended in 100 milliliters of acetonitrile and 25 grams of formic acid obtained from Aldrich Chemical (A.C.S. reagent grade). This was followed by the addition of 18 grams of formaldehyde (Aldrich, 37 weight % solution, A.C.S. reagent grade). The reaction mixture was heated to 60° C. for two hours, then filtered and washed twice with methanol, twice with 1% HCl in methanol/water (50/50) and then twice with water and then twice with 5% triethylmine in methanol, then finally twice with methanol. The product was bonded, methylated polyethyleneimine and was dried overnight at 70° C. in a convection oven and sieved to yield 12.9 grams of methylated product.

Synthesis of a Quaternized Ammonium Salt Functional Macromolecule From Methylated Polyethyleneimine Bonded to a Silica Surface

The methylated product was converted to a quaternary ammonium salt by suspending 10 grams of the product in 75 milliliters of dimethylformamide and 4.98 grams of sodium carbonate and 20 grams of methyl iodide. The reaction mixture was allowed to stand overnight, then filtered and washed twice with methanol and then six times with weak acetic acid solution until a pH of 5.0 was reached, and then twice more with methanol. The product, quaternary ammonium salt of polyethyleneimine bonded to silica was dried at 70° C. overnight in a convection oven and sieved to yield 8.7 grams of product.

### Example 2

### Copper Capture on the Non-Methylated, Bonded Polyethyleneimine Phase from the Above Synthesis Example.

A glass tube with a porous fit was constructed and 0.50 grams of the bonded, non-methylated polyethyleneimine phase was packed above the frit in the tube. Then, 0.01 M of CuSO₄ was passed through the column and the effluent was observed for column breakthrough. The Cu++ from the CuSO₄ solution was absorbed on the non-methylated polyethyleneimine material (0.011 grams of Cu++, 1.73×10⁻⁴ moles of Cu++, 2.15 weight % of the polyethyleneimine phase).

### Example 3

### Copper Capture on the Bonded, Methylated Polyethyleneimine Phase from the Above Synthesis Example

A glass tube with a porous frit was constructed and 0.50 grams of the bonded, methylated polyethyleneimine phase was packed above the frit in the tube. Then, 0.01 M of CuSO₄ was passed through the column and the effluent was observed for column breakthrough. The Cu++ from 18.6 milliliters of CuSO₄ was absorbed on the methylated polyethyleneimine phase (0.12 grams, 0.186 millimoles of Cu++, 2.35 weight % Cu++on the methylated polyethyleneimine phase, approximately 10% more capture than on the non-methylated polyethyleneimine phase).

### Example 4

### Dichromate Capture on the Bonded, Quaternized Polyethyleneimine Phase from the Above Synthesis Example.

A glass tube with a porous frit was constructed and 0.50 grams of the bonded, quaternized polyethyleneimine phase was packed above the frit in the glass tube. Then, 0.01 M of K₂Cr₂O₇ was passed through the column and the effluent was observed for column breakthrough. Cr₂O₇⁻² from 8.5 milliliters of K₂Cr₂O₇ was absorbed on the quaternized polyethyleneimine phase (0.018 grams, 0.085 millimole, 3.64 weight % Cr₂O₇⁻² on the quaternized polyethyleneimine phase).

### Example 5

### A Second Approach to Quaternizing Polyethyleneimine which has Been Bonded to Silica

Silica, 600 grams (PQ Corporation, MeS-3050, having a surface area of 490 m²/gram and a pore diameter of 261 Å and a particle size of 90 μ) was dispersed in 6 liters of toluene and azeotroped for two hours to remove surface moisture. Then 3.0 grams of glacial acetic acid was added followed by 288.5 grams of 3-glycidoxypropyl-trimethoxysilane. The mixture was allowed to sit overnight at 50° C. and then for three days at room temperature. The product was filtered and washed with toluene, then twice with methanol, and then suspended in N,N-dimethylformamide (EM Science, A.C.S. grade). Then 90 grams of polyethyleneimine (BASF, approximately 25,000 molecular weight) was added to the suspension and the mixture was allowed to react overnight at room temperature.

### Example 6

### The Use of Dry Bonding for Preparation of Silanized Silica

One kilogram of silica (PPG Flo-Gard SP Silica) having a surface area of 210 m/g and an average particle size of 30 μm was added to a large glass bottle along with 205.97 grams of 3-Glycidoxypropyltrimethoxysilane and the bottle was capped and the bottle was place on a roller for 2 hours at room temperature to thoroughly mix the materials. Then 10 grams of 1 weight % acetic acid in water was added and the bottle was returned to the roller for 3 days at room temperature and then placed in an oven at 50° C. overnight (approximately 16 hours). The material was washed twice with methanol and then re-suspended in 4 to 5 liters of dimethylformamide. There was added 20% polyethyleneimine in about 1 liter of dimethylformamide and the materials were allowed to react overnight at room temperature. The bottle was placed in a warm water bath for 2 hours, the material removed from the bottle and washed twice with methanol and then once with a mixture of methanol/HPLC water, and then twice with methanol, The treated silica/PEI material was dried in a convection oven at 70° C. overnight and then it was sieved and bottled.

### Example 7

### The Use of Dry Bonding for Preparation of Silanized Silica

The materials used in this example were silica obtained from PPG, Flo-Gard SP silica having a surface area of 210 m/g and a particle size of about 30 μm. Glycidoxypropyltrimethoxysilane, acetic acid, polyethyleneimine, dimethylformamide, methanol and HPLC grade water, the latter two materials being used for washing the samples.

The silica (1 kg.) was placed into a large bottle with 205.97 gms of the silane and the bottle was rolled on a roller for 2 hours, at room temperature. There was then added 10.00 gms of 1% acetic acid and the bottle was placed on the roller for 3 days, at room temperature. The bottle was then put into an oven at 50° C. for overnight (approximately 16 hours). The material was then washed twice with the methanol and re-suspended in about 4 to 5 liters of the dimethylformamide. Two hundred gms of the polyethyleneimine in about 1 liter of dimethylformamide was then added to the bottle and the mixture allowed to react for about 16 hrs. at room temperature. The bottle was then placed in warm water for about 2 hours and then the material in the bottle was washed twice with methanol and then once with methanol/water mixture, and then twice more with methanol. The product was dried in a convection oven 70° C. in an oven for about 16 hours and the material was then sieved and bottled.

What is claimed is:

1. An alkylated amine functional macromolecule comprising a base macromolecule covalently bonded to a solid substrate wherein the base macromolecule is selected from the group consisting of amine functional:
(a) dendrimers,
(b) hyperbranched polymers,
(c) multilayered dendrimers,
(d) multilayered hyperbranched polymers
(e) multilayered polymers of mixed dendrimer, and, hyperbranched multilayers.

2. An ammonium salt functional macromolecule comprising a base macromolecule covalently bonded to a solid substrate wherein the base macromolecule is selected from the group consisting of amine functional:
(a) dendrimers,
(b) hyperbranched polymers,
(c) multilayered dendrimers,
(d) multilayered hyperbranched polymers
(e) multilayered polymers of mixed dendrimer, and, hyperbranched multilayers.

3. A quaternized ammonium salt functional macromolecule comprising a base macromolecule covalently bonded to a solid substrate wherein the base macromolecule is selected from the group consisting of amine functional:
(a) dendrimers,
(b) hyperbranched polymers,
(c) multilayered dendrimers,
(d) multilayered hyperbranched polymers
(e) multilayered polymers of mixed dendrimer, and, hyperbranched multilayers.

4. A process for preparing an alkylated amine functional macromolecule, the process comprising:
(I) providing a solid substrate capable of reacting with a silane bonding agent;
(II) contacting the solid substrate with a silane bonding agent and allowing the solid substrate to react with the silane bonding agent;
(III) contacting the product formed in step (II) with a base macromolecule selected from the group consisting of amine functional:
(a) dendrimers,
(b) hyperbranched polymers,
(c) multilayered dendrimers,
(d) multilayered hyperbranched polymers
(e) multilayered polymers of mixed dendrimer, and, hyperbranched multilayers, allowing the base macromolecule and the product from step (II) to react with each other;
(IV) contacting the product from step (III) with formic acid and formaldehyde in a solvent at a temperature in excess of ambient temperature to form an alkylated macromolecule.

5. A process for preparing an ammonium salt functional macromolecule, the process comprising:
(I) providing a solid substrate capable of reacting with a silane bonding agent;
(II) contacting the solid substrate with a silane bonding agent and allowing the solid substrate to react with the silane bonding agent;
(III) contacting the product formed in step (II) with a base macromolecule selected from the group consisting of amine functional:
(a) dendrimers,
(b) hyperbranched polymers,
(c) multilayered dendrimers,
(d) multilayered hyperbranched polymers
(e) multilayered polymers of mixed dendrimer, and, hyperbranched multilayers.
and allowing the base macromolecule and the product from step (II) to react with each other;
(IV) contacting the product from step (III) with formic acid and formaldehyde in a solvent at a temperature in excess of ambient temperature to form an alkylated macromolecule;
(V) contacting the alkylated macromolecule with an acid for a period of time to form a macromolecule ammonium salt.

6. A process for preparing a quaternized ammonium salt functional macromolecule comprising:
(I) providing a solid substrate capable of reacting with a silane bonding agent;
(II) contacting the solid substrate with a silane bonding agent and allowing the solid substrate to react with the silane bonding agent;
(III) contacting the product formed in step (II) with a base macromolecule selected from the group consisting of amine functional:
(a) dendrimers,
(b) hyperbranched polymers,
(c) multilayered dendrimers,
(d) multilayered hyperbranched polymers
(e) multilayered polymers of mixed dendrimer, and, hyperbranched multilayers.
and allowing the base macromolecule and the product from step (II) to react with each other;
(IV) contacting the product from step (III) with formic acid and formaldehyde in a solvent at a temperature in excess of ambient temperature to form an alkylated macromolecule;
(V) contacting the alkylated macromolecule with a weak base and an excess of an alkyl halide in the presence of a solvent at ambient temperature or higher for a period of time to form a quaternized macromolecule.

7. A process for preparing an alkylated amine functional macromolecule comprising:
(I) providing a lot of base macromolecule selected from the group consisting of:
(a) dendrimers,
(b) hyperbranched polymers,
(c) multilayered dendrimers,
(d) multilayered hyperbranched polymers
(e) multilayered polymers of mixed dendrimer, and, hyperbranched multilayers.
(II) contacting the base macromolecule with a silane bonding agent and allowing the silane bonding agent and the base macromolecule to react with each other;
(III) contacting the product from step (II) with a solid substrate capable of reacting with the product from step (II);
(IV) allowing the product from step (III) to bond to the solid substrate by providing at least one of the conditions selected from the group consisting of:
i initially combining the components in step (II) in the absence of water and thereafter, contacting the combine components with water and,
ii initially combining the components in step (III) with sufficient water for hydrolysis of any hydrolysable groups in the components and thereafter, adding additional water for crosslinking any silanols formed by the initial water for hydrolysis;
(v) contacting the product from step (IV) with formic acid and formaldehyde in a quantity selected from the group consisting of:
i substoichiometric,
ii stoichiometric, and
iii excess stoichiometric based on the amount of reactive amine in the base macromolecule, in a solvent at a temperature selected from a range of from ambient temperature to higher than ambient temperature, to form an alkylated amine functional macromolecule.

8. A process for preparing an ammonium salt functional macromolecule, the process comprising:
(I) providing a lot of base macromolecule selected from the group consisting of:
(a) dendrimers,
(b) hyperbranched polymers,
(c) multilayered dendrimers,
(d) multilayered hyperbranched polymers
(e) multilayered polymers of mixed dendrimer, and, hyperbranched multilayers.
(II) contacting the base macromolecule with a silane bonding agent and allowing the silane bonding agent and the base macromolecule to react with each other;
(III) contacting the product from step (II) with a solid substrate capable of reacting with the product from step (II);
(IV) allowing the product from step (III) to bond to the solid substrate by providing at least one of the conditions selected from the group consisting of:
i initially combining the components in step (II) in the absence of water and thereafter, contacting the combine components with water and,
ii initially combining the components in step (III) with sufficient water for hydrolysis of any hydrolysable groups in the components and thereafter, adding additional water for crosslinking any silanols formed by the initial water for hydrolysis;
(V) contacting the product from step (IV) with formic acid and formaldehyde in a quantity selected from the group consisting of:
i substoichiometric,
ii stoichiometric, and
iii excess stoichiometric
based on the amount of reactive amine in the base macromolecule, in a solvent at a temperature selected from a range of from ambient temperature to higher than ambient temperature, to form an alkylated macromolecule;
(VI) contacting the alkylated macromolecule with an acid for a period of time to form an alkylated, macromolecule ammonium salt.

9. A process for preparing a quaternized ammonium salt functional macromolecule comprising:
(I) providing a lot of base macromolecule selected from the group consisting of:
(a) dendrimers,
(b) hyperbranched polymers,
(c) multilayered dendrimers,
(d) multilayered hyperbranched polymers
(e) multilayered polymers of mixed dendrimer, and, hyperbranched multilayers.
(II) contacting the base macromolecule with a silane bonding agent and allowing the silane bonding agent and the base macromolecule to react with each other;
(III) contacting the product from step (II) with a solid substrate capable of reacting with the product from step (II);
(IV) allowing the product from step (III) to bond to the solid substrate by providing at least one of the conditions selected from the group consisting of:
i initially combining the components in step (II) in the absence of water and thereafter, contacting the combine components with water and,
ii initially combining the components in step (III) with sufficient water for hydrolysis of any hydrolysable groups in the components and thereafter, adding additional water for crosslinking any silanols formed by the initial water for hydrolysis;
(V) contacting the product from step (IV) with formic acid and formaldehyde in a quantity selected from the group consisting of:
i substoichiometric,
ii stoichiometric, and
iii excess stoichiometric based on the amount of reactive amine in the base macromolecule, in a solvent at a temperature selected from a range of from ambient temperature to higher than ambient temperature, to form an alkylated macromolecule;
(VI) contacting the alkylated macromolecule with a weak base and a stoichiometric excess of an alkyl halide in the presence of a solvent at ambient temperature or higher for a period of time to form a quaternized macromolecule.

10. A method of analysis, the method comprising utilizing a composition as claimed in claim 1 for separating the desired material of analysis from materials associated with the desired material.

11. A method as claimed in claim 10 that is liquid chromatography, the method comprising utilizing the composition as a filler in a column employed in the liquid chromatography process.

12. A method of process stream purification, the method comprising treating a process stream effluent using a composition as claimed in claim 1.

13. A method as claimed in claim 12 wherein metal ions are removed from the process stream.

14. A method as claimed in claim 12 wherein acids are removed from the process stream.

15. A method of cleaning a waste stream, the method comprising treating a waste stream using a composition as claimed in claim 1.

16. A method of recovering resources, the method comprising treating a fluid containing said recoverable resources with a composition as claimed in claim 1, and thereafter, separating the fluid and the composition, and thereafter, recovering the recoverable resource from the composition.

17. A method as claimed in claim 16 wherein the fluid is a solvent, and the recoverable resource is a metal

18. A method of separating proteins, the method comprising utilizing a composition as claimed in claim 1 as the medium for separating the proteins from materials associated with the proteins.

19. An analytical method of separating peptides, the method comprising utilizing a composition as claimed in claim 1 to adsorb said peptides in preference to materials associated with the peptides.

20. A method of separating oligonucleotides from associated materials, the method comprising utilizing a composition of claim 1 to adsorb said oligonucleotides.

21. A method of analysis, the method comprising utilizing a composition as claimed in claim 2 for separating the desired material of analysis from materials associated with the desired material.

22. A method as claimed in claim 21 that is liquid chromatography, the method comprising utilizing the composition as a filler in a column employed in the liquid chromatography process.

23. A method of process stream purification, the method comprising treating a process stream effluent using a composition as claimed in claim 2.

24. A method as claimed in claim 23 wherein metal ions are removed from the process stream.

25. A method as claimed in claim 23 wherein acids are removed from the process stream.

26. A method of cleaning a waste stream, the method comprising treating a waste stream using a composition as claimed in claim 2.

27. A method of recovering resources, the method comprising treating a fluid containing said recoverable resources with a composition as claimed in claim 2, and thereafter, separating the fluid and the composition, and thereafter, recovering the recoverable resource from the composition.

28. A method as claimed in claim 27 wherein the fluid is a solvent, and the recoverable resource is a metal

29. A method of separating proteins, the method comprising utilizing a composition as claimed in claim 2 as the medium for separating the proteins from materials associated with the proteins.

30. An analytical method of separating peptides, the method comprising utilizing a composition as claimed in claim 2 to adsorb said peptides in preference to materials associated with the peptides.

31. A method of separating oligonucleotides from associated materials, the method comprising utilizing a composition of claim 2 to adsorb said oligonucleotides.

32. A method of analysis, the method comprising utilizing a composition as claimed in claim 3 for separating the desired material of analysis from materials associated with the desired material.

33. A method as claimed in claim 31 that is liquid chromatography, the method comprising utilizing the composition as a filler in a column employed in the liquid chromatography process.

34. A method of process stream purification, the method comprising treating a process stream effluent using a composition as claimed in claim 3.

35. A method as claimed in claim 34 wherein metal ions are removed from the process stream.

36. A method as claimed in claim 34 wherein acids are removed from the process stream.

37. A method of cleaning a waste stream, the method comprising treating a waste stream using a composition as claimed in claim 3.

38. A method of recovering resources, the method comprising treating a fluid containing said recoverable resources with a composition as claimed in claim 3, and thereafter, separating the fluid and the composition, and thereafter, recovering the recoverable resource from the composition.

39. A method as claimed in claim 38 wherein the fluid is a solvent, and the recoverable resource is a metal

40. A method of separating proteins, the method comprising utilizing a composition as claimed in claim 3 as the medium for separating the proteins from materials associated with the proteins.

41. An analytical method of separating peptides, the method comprising utilizing a composition as claimed in claim 3 to adsorb said peptides in preference to materials associated with the peptides.

42. A method of separating oligonucleotides from associated materials, the method comprising utilizing a composition of claim 3 to adsorb said oligonucleotides

43. A personal care product containing a composition of claim 1.

44. A personal care product containing a composition of claim 2.

45. A personal care product containing a composition of claim 3.

46. A household care product containing a composition of claim 1.

47. A household care product containing a composition of claim 2.

48. A household care product containing a composition of claim 3.

49. A health care product containing a composition of claim 1.

50. The health care product of claim 49 wherein the health care product is a topical health care product.

51. The health care product of claim 49 wherein the health care product is an in vivo health care product.

52. A health care product containing a composition of claim 2.

53. A health care product of claim 52 wherein the health care product is a topical health care product.

54. A health care product of claim 52 wherein the health care product is an in vivo health care product.

55. A health care product containing a composition of claim 3.

56. A health care product of claim 55 wherein the health care product is a topical health care product.

57. A health care product of claim 55 wherein the health care product is an in vivo health care product.
